(19) United States
(12) Patent Application Publication
(10) Pub. No.: US 2021/0153939 A1
(43) Pub. Date: May 27, 2021

(54) ENERGY MANIFOLD FOR DIRECTING AND CONCENTRATING ENERGY WITHIN A LITHOPLASTY DEVICE

(71) Applicant: Bolt Medical, Inc., Carlsbad, CA (US)

(72) Inventors: Christopher A. Cook, Laguna Niguel, CA (US); Eric Schultheis, San Clemente, CA (US)

(21) Appl. No.: 17/091,050

(22) Filed: Nov. 6, 2020

Related U.S. Application Data
(60) Provisional application No. 62/939,409, filed on Nov. 22, 2019.

(51) Int. Cl. A61B 18/24

(52) U.S. Cl. CPC A61B 18/245; A61B 2018/0022

(57) ABSTRACT

A catheter system for treating a vascular lesion within or adjacent to a vessel wall within a body of a patient includes a catheter fluid, an energy source that generates energy, an energy guide and an energy manifold. The energy guide includes a guide distal end that is selectively positioned near the vascular lesion. The energy guide is configured to receive energy from the energy source and generate a plasma bubble within the catheter fluid. The energy manifold is coupled to the energy guide near the guide distal end. The energy manifold includes (i) a manifold body that defines a body chamber, the body chamber being configured to retain at least some of the catheter fluid, and (ii) a manifold aperture that extends through the manifold body. The energy manifold directs energy from the plasma bubble out of the body chamber through the manifold aperture and toward the vascular lesion.

### RELATED APPLICATION

This application claims priority on U.S. Provisional Application Ser. No. 62/939,409, filed on Nov. 22, 2019, and entitled “ENERGY MANIFOLD FOR LASER-DRIVEN LITHOPLASTY DEVICE”. As far as permitted, the contents of U.S. Provisional Application Ser. No. 62/939,409 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

Lithoplasty is one method that has been recently used with some success for breaking up vascular lesions within vessels in the body. Lithoplasty utilizes a combination of pressure waves and bubble dynamics that are generated intravascularly in a fluid-filled balloon catheter. In particular, during a lithoplasty treatment, a high energy source is used to generate plasma and ultimately pressure waves as well as a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock, or water hammer.

It is desired to more accurately and precisely direct and/or concentrate energy generated within the fluid-filled balloon so as to impart pressure onto and induce fractures in vascular lesions at a treatment site within or adjacent to a blood vessel wall.

There is an ongoing desire to enhance vessel patency and optimization of therapy delivery parameters within a lithoplasty catheter system.

### SUMMARY

The present invention is directed toward a catheter system for placement within a blood vessel having a vessel wall. The catheter system can be used for treating a vascular lesion within or adjacent to the vessel wall within a body of a patient. The catheter system includes a catheter fluid and an energy source that generates energy. In various embodiments, the catheter system includes an energy guide and an energy manifold. The energy guide includes a guide distal end that is selectively positioned near the vascular lesion. The energy guide is configured to receive energy from the energy source and generate a plasma bubble within the catheter fluid. The energy manifold is coupled to the energy guide near the guide distal end. The energy manifold includes (i) a manifold body that defines a body chamber, the body chamber being configured to retain at least some of the catheter fluid, and (ii) a manifold aperture that extends through the manifold body. The energy manifold directs energy from the plasma bubble out of the body chamber through the manifold aperture and toward the vascular lesion.

In some embodiments, the energy manifold includes a plurality of manifold apertures that extend through the manifold body. In such embodiments, the energy manifold is configured to direct energy from the plasma bubble out of the body chamber through each of the plurality of manifold apertures and toward the vascular lesion. In one such embodiment, the plurality of manifold apertures are positioned in a radial pattern around a perimeter of the manifold body. In another such embodiment, the plurality of manifold apertures are arranged in a spiral pattern along a length of the manifold body. In still another such embodiment, the plurality of manifold apertures are positioned along a length of the manifold body.

In certain embodiments, the energy guide generates one or more pressure waves within the catheter fluid that impart a force upon the vascular lesion. Further, the energy guide can include an optical fiber.

In some embodiments, the catheter system further includes a balloon including a balloon wall that defines a balloon interior. The balloon is configured to retain the catheter fluid within the balloon interior. The guide distal end and the energy manifold are positioned within the balloon interior. In certain such embodiments, the balloon is selectively inflatable with the catheter fluid to expand to an inflated state. When the balloon is in the inflated state, the balloon wall is configured to be positioned substantially adjacent to the vascular lesion. Moreover, in some such embodiments, the energy manifold is configured to direct energy from the plasma bubble out of the body chamber through the manifold aperture and toward the balloon wall.

In certain embodiments, the manifold body includes a manifold proximal end, and the guide distal end of the energy guide is secured to the manifold proximal end of the manifold body.

In one embodiment, the manifold body is substantially cylindrical tube-shaped and defines a substantially cylindrical-shaped body chamber. In another embodiment, the manifold body includes the manifold proximal end and an opposed manifold distal end, and the body chamber is tapered such that the body chamber is larger near the manifold proximal end and smaller near the manifold distal end.

In some embodiments, the catheter system further includes a guide end protector that is coupled to the guide distal end, the guide end protector being configured to protect the guide distal end from energy from the plasma bubble that is generated in the body chamber.

In certain embodiments, the energy manifold further includes an energy diverter that diverts energy from the plasma bubble that is generated in the body chamber toward the manifold aperture. In some such embodiments, the manifold body includes a manifold distal end, and the energy diverter is positioned adjacent to the manifold distal end.

In some embodiments, the energy manifold further includes an optical element that is configured to focus the energy that is directed from the guide distal end of the energy guide. In one embodiment, the optical element is formed from sapphire, although it is appreciated that the optical element can be formed from other suitable materials. In alternative embodiments, the optical element can be directly coupled to the guide distal end of the energy guide, the optical element can be formed directly onto the guide distal end of the energy guide, or the optical element can be positioned spaced apart from the guide distal end of the energy guide to define an air space between the guide distal end and the optical element. In certain embodiments, the air space is sealed from the remainder of the body chamber such that no catheter fluid is retained within the air space.

In certain embodiments, the catheter system further includes a guide endcap that is directly coupled to the guide distal end of the energy guide. In such embodiments, the optical element can be directly coupled to the guide endcap. Further, in some such embodiments, at least one of the guide endcap and the optical element is formed from glass. Still further, in certain embodiments, the manifold body includes a manifold proximal end, and the manifold proximal end is secured to the optical element.

In some embodiments, the catheter fluid includes one of a wetting agent and a surfactant.

In certain embodiments, the catheter system further includes an extension tube that is coupled to and extends away from the guide distal end of the energy guide, the extension tube being configured to retain at least some of the catheter fluid. In such embodiments, the energy from the energy source is transmitted through the extension tube after being guided through the energy guide.

The present invention is further directed toward a method for treating a vascular lesion within or adjacent to a vessel wall within a body of a patient, the method including the steps of (A) generating energy with an energy source; (B) positioning a guide distal end of an energy guide near the vascular lesion; (C) coupling an energy manifold to the energy guide near the guide distal end, the energy manifold including (i) a manifold body that defines a body chamber, the body chamber being configured to retain at least some of a catheter fluid, and (ii) a manifold aperture that extends through the manifold body; (D) receiving energy from the energy source with the energy guide; (E) generating a plasma bubble within the catheter fluid with the energy from the energy guide; and (F) directing energy from the plasma bubble with the energy manifold out of the body chamber through the manifold aperture and toward the vascular lesion.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments, the catheter system including an energy guide and an energy manifold;

FIG. 2 is a schematic cross-sectional view of a portion of an embodiment of the catheter system including an embodiment of the energy manifold;

FIG. 3 is a schematic cross-sectional view of a portion of the energy guide and another embodiment of the energy manifold;

FIG. 4 is a schematic cross-sectional view of a portion of the energy guide and still another embodiment of the energy manifold;

FIG. 5 is a schematic cross-sectional view of a portion of the energy guide and yet another embodiment of the energy manifold;

FIG. 6 is a schematic cross-sectional view of a portion of the energy guide and still another embodiment of the energy manifold;

FIG. 7 is a schematic cross-sectional view of a portion of the energy guide and yet another embodiment of the energy manifold;

FIG. 8 is a schematic cross-sectional view of a portion of the energy guide and still yet another embodiment of the energy manifold;

FIG. 9A is a schematic cross-sectional view of an alternative embodiment of an energy guide assembly usable within the catheter system; and

FIG. 9B is a schematic cross-sectional view of another alternative embodiment of the energy guide assembly.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In certain embodiments, the catheter systems and related methods utilize an energy source, e.g., a light source such as a laser source or another suitable energy source, which provides energy that is guided by one or more energy guides, e.g., light guides such as optical fibers, which are disposed along the catheter shaft and within the balloon interior of the balloon to create a localized plasma in the catheter fluid that is retained within the balloon interior of the balloon. As such, the energy guide can sometimes be referred to as, or can be said to incorporate a “plasma generator” at or near a guide distal end of the energy guide that is positioned within the balloon interior of the balloon located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles (also sometimes referred to simply as “plasma bubbles”) can generate one or more pressure waves within the catheter fluid retained within the balloon interior of the balloon and thereby impart pressure waves onto and induce fractures in the vascular lesions at the treatment site within or adjacent to the blood vessel wall within the body of the patient. In some embodiments, the energy source can be configured to provide sub-millisecond pulses of energy, e.g., light energy, to initiate the plasma formation in the catheter fluid within the balloon to cause the rapid bubble formation and to impart the pressure waves upon the balloon wall at the treatment site. Thus, the pressure waves can transfer mechanical energy through an incompressible catheter fluid to the treatment site to impart a fracture force on the intravascular lesion. Without wishing to be bound by any particular theory, it is believed that the rapid change in catheter fluid momentum upon the balloon wall that is in contact with the intravascular lesion is transferred to the intravascular lesion to induce fractures to the lesion.

Importantly, the catheter systems and related methods disclosed herein further include an energy manifold that is positioned within the balloon and that is coupled to and/or secured to the energy guide. The energy manifold is configured to direct and/or concentrate energy generated within the catheter fluid that is retained within the balloon, and is at least partially retained within the energy manifold, so as to impart pressure onto and induce fractures in the vascular lesion at the treatment site within or adjacent to the blood vessel. More particularly, the energy manifold directs and/or concentrates acoustic and mechanical energy produced by a lithoplasty device, such as a laser-driven pressure wave generating device, to impart pressure onto and induce fractures in the vascular lesion at the treatment site within or adjacent to the blood vessel within the body of the patient.

As used herein, the terms “intravascular lesion” and “vascular lesion” are used interchangeably unless otherwise noted. As such, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

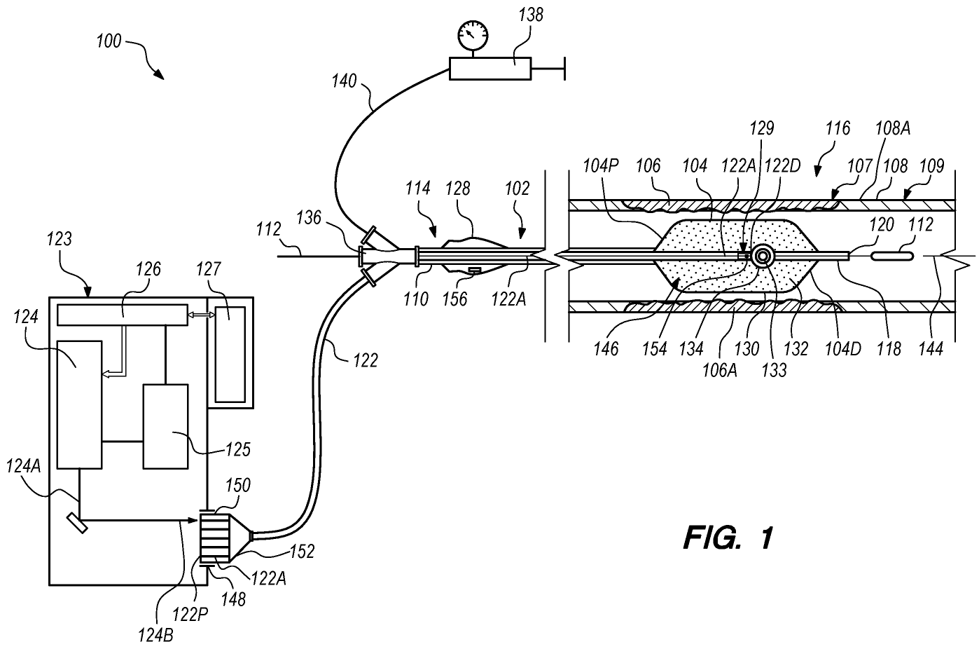

The catheter systems disclosed herein can include many different forms. Referring now to FIG. 1, a schematic cross-sectional view is shown of a catheter system 100 in accordance with various embodiments. The catheter system 100 is suitable for imparting pressure waves to induce fractures in one or more vascular lesions within or adjacent a vessel wall of a blood vessel. In the embodiment illustrated in FIG. 1, the catheter system 100 can include one or more of a catheter 102, an energy guide bundle 122 including one or more energy guides 122A, a source manifold 136, a fluid pump 138, a system console 123 including one or more of an energy source 124, a power source 125, a system controller 126, and a graphic user interface 127 (a “GUI”), a handle assembly 128, and an energy manifold 129. Alternatively, the catheter system 100 can have more components or fewer components than those specifically illustrated and described in relation to FIG. 1.

The catheter 102 is configured to move to a treatment site 106 within or adjacent to a vessel wall 108A of a blood vessel 108 within a body 107 of a patient 109. The treatment site 106 can include one or more vascular lesions 106A such as calcified vascular lesions, for example. Additionally, or in the alternative, the treatment site 106 can include vascular lesions 106A such as fibrous vascular lesions.

The catheter 102 can include an inflatable balloon 104 (sometimes referred to herein simply as a “balloon”), a catheter shaft 110 and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118 which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 when in the inflated state, this is done for ease of illustration. It is recognized that the balloon wall 130 of the balloon 104 will typically be substantially directly adjacent to and/or abutting the treatment site 106 when the balloon 104 is in the inflated state.

The balloon 104 suitable for use in the catheter system 100 includes those that can be passed through the vasculature of a patient when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as the energy manifold(s) 129). This reduces or eliminates the accumulation of bubbles and pockets or inclusions of gas within the energy manifold 129. Nonexclusive examples of chemicals that can be used as wetting agents include, but are not limited to, Benzalkonium Chloride, Benzethonium Chloride, Cetylpyridinium Chloride, Poloxamer 188, Poloxamer 407, Polysorbate 20, Polysorbate 40, and the like. Non-exclusive examples of surfactants can include, but are not limited to, ionic and non-ionic detergents, and Sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

By using a wetting agent or surfactant, direct liquid contact with the energy guide 122A allows the energy to be more efficiently converted into a plasma. Further, using the wetting agent or surfactant with the small dimensions of the optical and mechanical components used in the energy manifold 129 and other parts of the catheter 102, it is less difficult to achieve greater (or complete) wetting. Decreasing the surface tension of the liquid can decrease the difficulty for such small structures to be effectively wetted by the liquid and therefore be nearly or completely immersed. By reducing or eliminating air or other gas bubbles from adhering to the optical and mechanical structure and energy guides 122A, considerable increase in efficiency of the device can occur.

The specific percentage of the wetting agent or surfactant can be varied to suit the design parameters of the catheter system 100 and/or the energy manifold 129 being used. In one embodiment, the percentage of the wetting agent or surfactant can be less than approximately 50% by volume of the catheter fluid 132. In non-exclusive alternative embodiments, the percentage of the wetting agent or surfactant can be less than approximately 40%, 30%, 20%, 10%, 5%, 2%, 1%, 0.1% or 0.01% by volume of the catheter fluid 132. Still alternatively, the percentage of the wetting agent or surfactant can fall outside of the foregoing ranges.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. In some embodiments, each energy guide 122A can be an optical fiber and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A such as a first energy guide, a second energy guide, a third energy guide, etc., which can be disposed at any suitable positions about the guidewire lumen 118 and/or the catheter shaft 110. For example, in certain non-exclusive embodiments, two energy guides 122A can be spaced apart by approximately 180 degrees about the circumference of the guidewire lumen 118 and/or the catheter shaft 110; three energy guides 122A can be spaced apart by approximately 120 degrees about the circumference of the guidewire lumen 118 and/or the catheter shaft 110; or four energy guides 122A can be spaced apart by approximately 90 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A.

The energy guides 122A can have any suitable design for purposes of generating plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves within the catheter fluid 132 that are utilized to provide the fracture force onto the vascular lesions 106A at the treatment site 106. Still alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D having at least one optical window (not shown) that is positioned within the balloon interior 146.

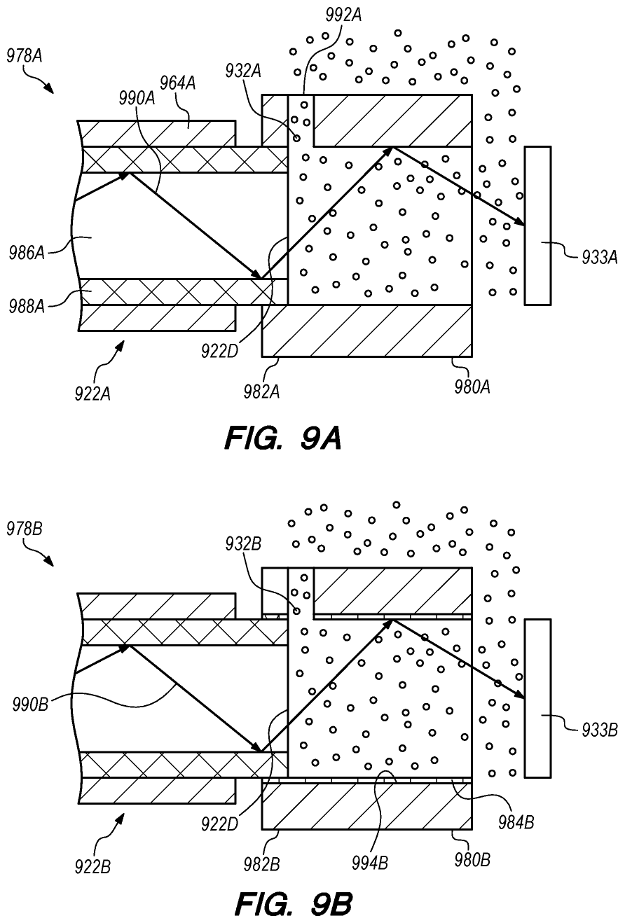

Alternatively, the energy guides 122A can have another suitable design and/or the energy from the energy source 124 can be guided into the balloon interior 146 by another suitable method. For example, in some non-exclusive alternative embodiments, guiding of the energy from the energy source 124 into the balloon interior 146 can be performed with an energy guide assembly 978A (illustrated in FIG. 9A) that can include an energy guide 122A similar to those described in various embodiments, and an extension tube 980A (illustrated in FIG. 9A) that is coupled to and/or secured to the guide distal end 122D of the energy guide 122A. In such embodiments, the extension tube 980A can be a hollow tube that is configured to be filled with the catheter fluid 132. In certain such embodiments, the extension tube 980A can include tube walls 982A (illustrated in FIG. 9A) having an index of refraction that is lower than the index of refraction of the catheter fluid 132 that can be retained within the extension tube 980A. Additionally, in alternative such embodiments, the extension tube 980A can be formed from a polymeric material, or the extension tube 980A can include a rigid and/or metallic substrate with a dielectric coating 984B (illustrated in FIG. 9B) that is provided on an inner surface of the extension tube 980A. Some such alternative embodiments will be described in greater detail in relation to FIGS. 9A and 9B.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. Additionally, in such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A can further include one or more diverting features or “diverters” (not shown in FIG. 1) within the energy guide 122A that are configured to direct energy to exit the energy guide 122A toward a side surface which can be located at or near the guide distal end 122D of the energy guide 122A, and toward the balloon wall 130. A diverting feature can include any feature of the system that diverts energy from the energy guide 122A away from its axial path toward a side surface of the energy guide 122A. Additionally, the energy guides 122A can each include one or more optical windows disposed along the longitudinal or circumferential surfaces of each energy guide 122A and in optical communication with a diverting feature. Stated in another manner, the diverting features can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting features suitable for use include a reflecting element, a refracting element, and a fiber diffuser. The diverting features suitable for focusing energy away from the tip of the energy guides 122A can include, but are not to be limited to, those having a convex surface, a gradient-index (GRIN) lens, and a mirror focus lens. Upon contact with the diverting feature, the energy is diverted within the energy guide 122A to one or more of a plasma generator 133 and the photoacoustic transducer 154 that is in optical communication with a side surface of the energy guide 122A. The photoacoustic transducer 154 then converts light energy into an acoustic wave that extends away from the side surface of the energy guide 122A.

Additionally, or in the alternative, in certain embodiments, diverting features that can be incorporated into the energy guides 122A, can also be incorporated into the design of the energy manifold 129 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132, i.e. via the inflation conduit 140, as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, e.g., the guide proximal end 122P, of each of the energy guides 122A. The guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 during use of the catheter system 100.

The energy source 124 can be selectively and/or alternatively coupled in optical communication with each of the energy guides 122A, i.e. to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, e.g., via the plasma generator 133 that can be located at the guide distal end 122D of the energy guide 122A. In particular, the energy emitted at the guide distal end 122D of the energy guide 122A energizes the plasma generator 133 to form the plasma within the catheter fluid 132 within the balloon interior 146. The plasma formation causes rapid bubble formation, and imparts pressure waves upon the treatment site 106. An exemplary plasma-induced bubble 134 is illustrated in FIG. 1.

In various non-exclusive alternative embodiments, the sub-millisecond pulses of energy from the energy source 124 can be delivered to the treatment site 106 at a frequency of between approximately one hertz (Hz) and 5000 Hz, approximately 30 Hz and 1000 Hz, approximately ten Hz and 100 Hz, or approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, i.e. a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 is electrically coupled to and receives power from the power source 125. Additionally, the system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control operation of other components of the catheter system 100 such as the positioning of the catheter 102 adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. Additionally, the GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, e.g., within the system console 123. It is understood that the handle assembly 128 can include fewer or additional components than those specifically illustrated and described herein.

The energy manifold 129 is configured to direct and/or concentrate energy generated within the catheter fluid 132 within the balloon interior 146 so as to impart pressure onto and induce fractures in vascular lesions 106A at the treatment site 106 within or adjacent to a vessel wall 108A of a blood vessel 108. More particularly, the energy manifold 129 is configured to concentrate and direct acoustic and/or mechanical energy toward specific areas of the balloon wall 130 in contact with the vascular lesions 106A at the treatment site 106 to enhance the delivery of such energy to the treatment site 106. Thus, the energy manifold 129 is able to effectively improve the efficacy of the catheter system 100.

It is appreciated that, in some embodiments, a separate energy manifold 129 can be included with and/or incorporated into each individual energy guide 122A. Alternatively, in other embodiments, a single energy manifold 129 can be configured to operate in conjunction with more than one energy guide 122A. Still alternatively, each energy guide 122A need not have an energy manifold 129 incorporated therein or associated therewith.

The design of the energy manifold 129 and/or the specific positioning of the energy manifold 129 can be varied to suit the requirements of the catheter system 100. In various embodiments, the energy manifold 129 can be coupled and/or secured to the energy guide 122A, i.e. at or near a guide distal end 122D of the energy guide 122A. Alternatively, the energy manifold 129 can be separated and/or spaced apart from the energy guide 122A.

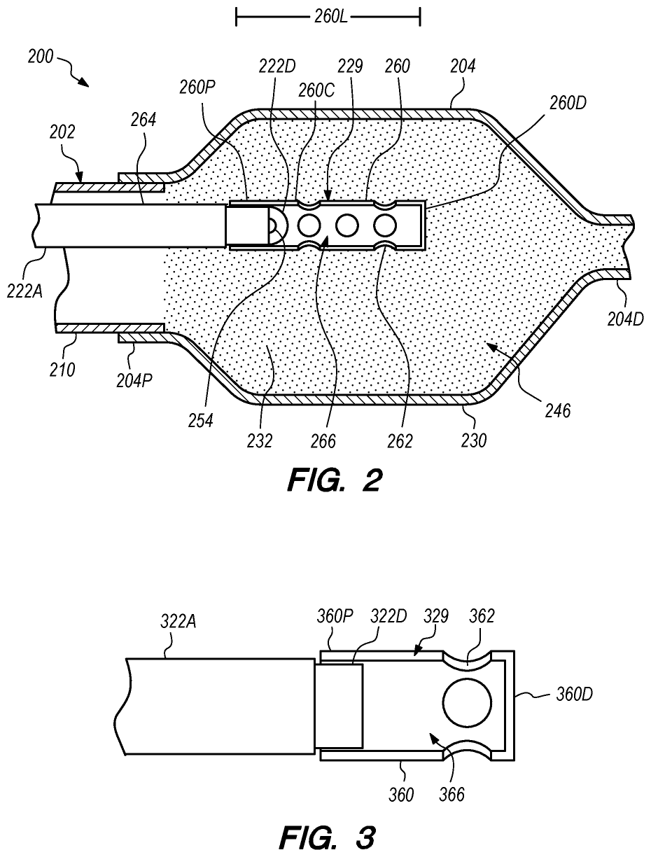

In certain embodiments, the energy manifold 129 can include a manifold body 260 (illustrated, for example, in FIG. 2), and one or more manifold apertures 262 (illustrated, for example, in FIG. 2) that are positioned within and/or extend through the manifold body 260 to direct the acoustic and/or mechanical energy in the form of the plasma that has been generated within the catheter fluid 132 toward the balloon wall 130 positioned adjacent to the treatment site 106. The one or more manifold apertures 262 can be provided in any suitable size, shape, orientation and pattern in order to direct the acoustic and/or mechanical energy as desired. For example, in some embodiments, the manifold apertures 262 can be round, square, rectangular, triangular, or have other suitable shapes specifically engineered to direct and concentrate the acoustic and/or mechanical energy to specific locations within the balloon 104.

Additionally, the energy manifold 129 can include any suitable number of manifold apertures 262. For example, in certain embodiments, the energy manifold 129 includes only a single manifold aperture 262 that can be positioned anywhere on, within, or along the manifold body 260 of the energy manifold 129. Alternatively, in other embodiments, the energy manifold 129 can include a plurality of manifold apertures 262, e.g., two, three, four, or more than four manifold apertures 262, which can be positioned in any suitable pattern on, within, or along the manifold body 260 of the energy manifold 129. In one non-exclusive such embodiment, the manifold apertures 262 can be positioned in a radial pattern around a circumference of the energy manifold 129. In another non-exclusive such embodiment, the manifold apertures 262 can be arranged in a spiral pattern running along a length of the energy manifold 129. In still another non-exclusive such embodiment, the manifold apertures 262 can be staggered along the length of the energy manifold 129 so as to emit in alternating directions. Alternatively, the manifold apertures 262 can be arranged in another suitable manner on, within, or along the manifold body 260 of the energy manifold 129.

Various alternative embodiments of the energy manifold 129 are illustrated and described in detail herein below within subsequent Figures.

FIG. 2 is a schematic cross-sectional view of a portion of an embodiment of the catheter system 200, including an embodiment of the energy manifold 229. The design of the catheter system 200 can be varied. In various embodiments, as illustrated in FIG. 2, the catheter system 200 can include a catheter 202 including a catheter shaft 210; a balloon 204 having a balloon wall 230 that defines a balloon interior 246, a balloon proximal end 204P, and a balloon distal end 204D; and a catheter fluid 232 that is retained substantially within the balloon interior 246; an energy guide 222A; and the energy manifold 229. Alternatively, in other embodiments, the catheter system 200 can include more components or fewer components than what is specifically illustrated and described herein. For example, certain components that were illustrated in FIG. 1, e.g., the guidewire 112, the guidewire lumen 118, the source manifold 136, the fluid pump 138, the energy source 124, the power source 125, the system controller 126, the GUI 127, and the handle assembly 128, are not specifically illustrated in FIG. 2 for purposes of clarity, but would likely be included in any embodiment of the catheter system 200.

The design and function of the catheter shaft 210, the balloon 204, the catheter fluid 232, and the energy guide 222A are substantially similar to what was illustrated and described herein above. Accordingly, a detailed description of such components will not be repeated.

The balloon 204 is again selectively movable between a deflated state suitable for advancing the catheter 202 through a patient's vasculature, and an inflated state suitable for anchoring the catheter 202 in position relative to the treatment site 106 (illustrated in FIG. 1). In some embodiments, the balloon proximal end 204P can be coupled to the catheter shaft 210, and the balloon distal end 204D can be coupled to the guidewire lumen 118 (illustrated in FIG. 1). The balloon 204 can again be inflated with the catheter fluid 232, e.g., from the fluid pump 138 (illustrated in FIG. 1), that is directed into the balloon interior 246 of the balloon 204 via the inflation conduit 140 (illustrated in FIG. 1).

Similar to previous embodiments, the energy guide 222A can include one or more photoacoustic transducers 254 (only one photoacoustic transducer 254 is illustrated in FIG. 2), where each photoacoustic transducer 254 can be in optical communication with the energy guide 222A within which it is disposed. In some embodiments, the photoacoustic transducers 254 can be in optical communication with the guide distal end 222D of the energy guide 222A. Additionally, in such embodiments, the photoacoustic transducers 254 can have a shape that corresponds with and/or conforms to the guide distal end 222D of the energy guide 222A. The photoacoustic transducer 254 is configured to convert light energy into an acoustic wave at or near the guide distal end 222D of the energy guide 222A. The direction of the acoustic wave can be tailored by changing an angle of the guide distal end 222D of the energy guide 222A.

In various embodiments, the energy manifold 229 is configured to direct and/or concentrate energy generated in the catheter fluid 232 within the balloon interior 246 to impart pressure onto and induce fractures in vascular lesions 106A (illustrated in FIG. 1) at the treatment site 106. More particularly, the energy manifold 229 is configured to direct and concentrate acoustic and/or mechanical energy toward specific areas of the balloon wall 230 that are in contact with the vascular lesions 106A at the treatment site 106 to enhance the delivery of such energy to the treatment site 106. Further, as illustrated in this embodiment, the energy manifold 229 is positioned inside the balloon 204 that can be filled with the catheter fluid 232.

As shown in the embodiment illustrated in FIG. 2, the energy manifold 229 is coupled to and/or secured to the energy guide 222A. Alternatively, the energy manifold 229 can be separated and/or spaced apart from the energy guide 222A.

The design of the energy manifold 229 can be varied. In certain embodiments, as shown in FIG. 2, the energy manifold 229 includes a manifold body 260 and one or more manifold apertures 262 that are positioned within and/or extend through the manifold body 260 to direct energy in the form of the plasma that has been generated within the catheter fluid 232 toward the balloon wall 230 positioned adjacent to the treatment site 106. In particular, the one or more manifold apertures 262 are configured such that the energy generated within the catheter fluid 232 through use of the energy guide 222A is directed outwardly, e.g., radially, away from the energy guide 222A and the energy manifold 229 and toward the balloon wall 230. The energy manifold 229 and/or the manifold apertures 262 can further be configured and/or positioned to direct and concentrate energy in a manner to most effectively impart pressure onto and induce fractures in vascular lesions 106A at precise locations within the treatment site 106 within or adjacent to a blood vessel wall. Additionally, or in the alternative, the energy manifold 229 can include more components than what is specifically illustrated in FIG. 2. In many embodiments, the energy manifold 229 can further include certain other features that further impact the overall operation of the energy manifold 229 and can thus improve the overall efficacy of the catheter system 200. For example, in other embodiments, the energy manifold 229 can include one or more of a guide end protector, an energy diverter, and an optical element that can be utilized to more effectively concentrate and direct the energy as desired through the manifold apertures 262 and toward the desired locations within the treatment site 106.

The manifold body 260 and the manifold apertures 262 can have any suitable design, size, shape and orientation. In its simplest form the manifold body 260 is provided in the form of a perforated, elongated, cylindrical tube, including the one or more manifold apertures 262 as the noted perforations strategically positioned within and/or extending through the manifold body 260. As shown in the embodiment illustrated in FIG. 2, the manifold apertures 262 can be positioned in a radial pattern around a perimeter 260C, or circumference, of the manifold body 260. Additionally, or in the alternative, the manifold apertures 262 can be positioned in another suitable manner relative to the manifold body 260. For example, in certain non-exclusive embodiments, the manifold apertures 262 can also be positioned spaced apart from one another along a length 260L of the manifold body 260 and/or the manifold apertures 262 can be arranged in a spiral pattern running along the length 260L of the manifold body 260. Alternatively, the manifold body 260 can have another suitable design and/or the manifold apertures 262 can be positioned in another suitable manner.

As illustrated in FIG. 2, the energy guide 222A can be located at or near a manifold proximal end 260P of the manifold body 260, i.e. with the guide distal end 222D of the energy guide 222A inserted into the manifold proximal end 260P of the elongated manifold body 260. As shown in the embodiment illustrated in FIG. 2, the energy guide 222A can have be a generally semi-spherical, ball-shaped guide distal end 222D through which energy is directed out of the energy guide 222A. Alternatively, the guide distal end 222D can have another suitable shape, such as a flat, cleaved end, or any other suitable shape. In some embodiments, the energy guide 222A can be secured, e.g., directly secured, to the manifold body 260. The energy guide 222A can be secured to the manifold body 260 in any suitable manner. However, it is appreciated that the energy guide 222A need not be directly secured to the manifold body 260. In certain embodiments, the energy guide 222A can include a guide jacket 264 that is configured to surround and protect the energy guide 222A along a substantial length of the energy guide 222A.

As shown, the manifold body 260 defines a substantially cylindrical-shaped, body chamber 266 (or “body cavity”) that extends away from the guide distal end 222D of the energy guide 222A and toward a manifold distal end 260D of the manifold body 260. Alternatively, the manifold body 260 can define a body chamber 266 having another suitable shape, e.g., with a somewhat tapered design, with segmented chambers, and/or with a body chamber 266 that is other than generally cylindrical-shaped.

During use of the catheter system 200, the catheter fluid 232 that is utilized to inflate the balloon 204 also is allowed to enter from the balloon interior 246 into at least a portion of the body chamber 266 as defined by the manifold body 260 through the one or more manifold apertures 262. Subsequently, the pulsed energy that is directed through the energy guide 222A generates a plasma-induced bubble 134 (illustrated in FIG. 1) ahead of the guide distal end 222D and within the catheter fluid 232 that is present within the body chamber 266 of the energy manifold 229. As the bubble 134 expands, it drives the catheter fluid 232 ahead of it down the length of the body chamber 266. Thus, the expanding bubble 134 is directed through the body chamber 266, and is allowed to escape selectively as it passes by and/or through the manifold apertures 262 that are formed into and extend through the manifold body 260. As such, the manifold apertures 262 direct the energy from the plasma-induced bubble 134 outward toward the balloon wall 230 and concentrate the energy, e.g., the acoustic energy from the photoacoustic transducer 254, delivered there.

In this embodiment, the manifold distal end 260D is substantially flat, and the manifold distal end 260D is sealed such that it blocks and redirects energy that is generated within the body chamber 266, e.g., any energy that initially passes by the manifold apertures 262 within the body chamber 266, back toward the manifold apertures 262. Thus, the energy can be more effectively directed through the manifold apertures 262 and toward the balloon wall 230 adjacent the treatment site 106.

With such design, the energy created by one energy guide 222A can be distributed through a long, narrow balloon 204 of the catheter assembly 200, and can be directed, e.g., radially, through the manifold apertures 262 and toward the balloon wall 230. The energy from one energy guide 222A and/or one energy source 124, especially in balloons 204 of greater length, can therefore treat multiple regions of the treatment site 106 (or multiple treatment sites 106) simultaneously.

It is appreciated that the manifold apertures 262 can vary in size, shape and orientation in order to distribute energy evenly along the length 260L of the manifold body 260 as energy in the bubble 134 itself is dissipated with propagation distance. For example, in some embodiments, the manifold apertures 262 can be smaller towards the manifold proximal end 260P of the manifold body 260 and increase in cross-sectional area towards the manifold distal end 260D of the manifold body 260. In different non-exclusive embodiments, the manifold apertures 262 can be substantially circle-shaped, oval-shaped, square-shaped, rectangle-shaped, or another suitable shape.

The manifold body 260 can include any suitable number of manifold apertures 262 in order that the energy is directed as desired toward the vascular lesion(s) at the treatment site 106.

FIG. 3 is a schematic cross-sectional view of a portion of the energy guide 322A and another embodiment of the energy manifold 329. As shown in this embodiment, the energy manifold 329 is substantially similar in design, positioning and function to the energy manifold 229 illustrated and described in relation to the FIG. 2. For example, the energy manifold 329 again includes a manifold body 360 including a manifold proximal end 360P that is coupled to and/or secured to the guide distal end 322D of the energy guide 322A and a substantially flat, sealed, manifold distal end 360D; and one or more manifold apertures 362 that are formed into and/or extend through the manifold body 360. In this embodiment, the energy manifold 329 is again configured to direct and concentrate acoustic and/or mechanical energy from the body chamber 366 as defined by the manifold body 360 through the manifold apertures 362 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106.

However, in this embodiment, the guide distal end 322D of the energy guide 322A has a slightly different shape than in the previous embodiment. In particular, as shown in FIG. 3, the energy guide 322A can have a flat, cleaved guide distal end 322D through which energy is directed out of the energy guide 322A and into the body chamber 366, instead of a generally semi-spherical, ball-shaped end as was shown in the previous embodiment. In non-exclusive alternative embodiments, the shape of the guide distal end 322D can be conical, wedge-shaped or pyramidal. Still alternatively, the shape of the guide distal end 322D can have any other suitable geometry, shape or configuration.

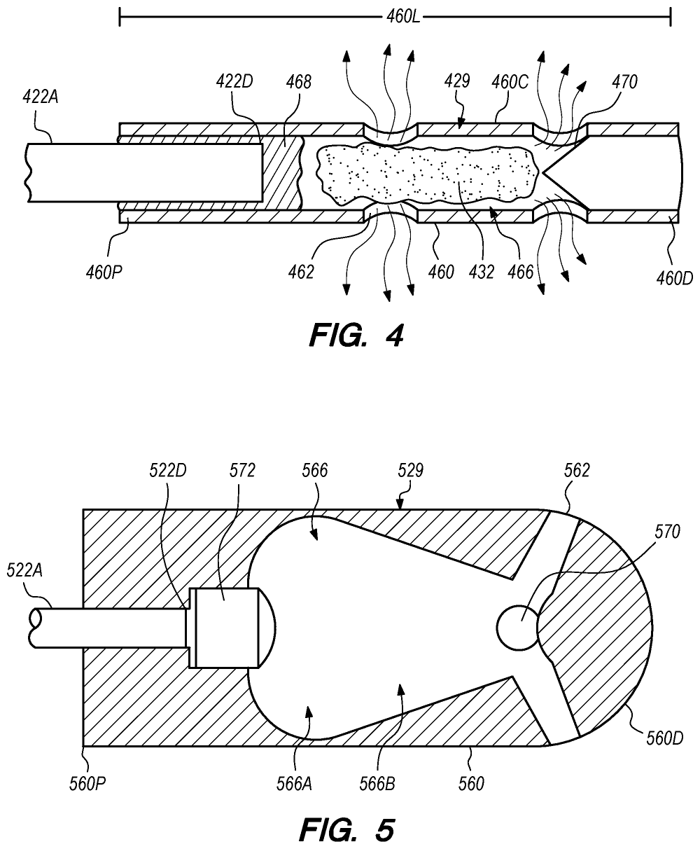

FIG. 4 is a schematic cross-sectional view of a portion of the energy guide 422A and still another embodiment of the energy manifold 429. As shown in FIG. 4, the energy manifold 429 is somewhat similar in design, positioning and function to the previous embodiments. For example, the energy manifold 429 again includes a manifold body 460 including a manifold proximal end 460P that is coupled to and/or secured to the guide distal end 422D of the energy guide 422A; and one or more manifold apertures 462 that are formed into and/or extend through the manifold body 460, i.e. at various points along a length 460L of the manifold body 460 and/or about a perimeter 460C of the manifold body 460. In this embodiment, the energy manifold 429 is again configured to direct and concentrate acoustic and/or mechanical energy from the body chamber 466 as defined by the manifold body 460 through the manifold apertures 462 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106. It is appreciated that at least a portion of the catheter fluid 432 and/or plasma that is positioned and/or generated within the body chamber 466 of the manifold body 460 is also illustrated in FIG. 4

However, as shown in the embodiment illustrated in FIG. 4, the energy manifold 429 and/or the energy guide 422A further includes a guide end protector 468, and an energy diverter 470.

The guide end protector 468 is coupled to the guide distal end 422D of the energy guide 422A. The guide end protector 468 is configured to at least substantially completely surround or encircle the guide distal end 422D to protect the guide distal end 422D from the plasma and pressure waves that are generated within the catheter fluid 232 (illustrated in FIG. 2). However, the guide end protector 468 is formed in such manner that energy is still able to be emitted from the guide distal end 422D of the energy guide 422A as desired. The guide end protector 468 can have any suitable design and/or can be formed from any suitable materials. For example, in certain non-exclusive embodiments, the guide end protector 468 can include and/or be formed from one or more of silicone, polymethyl methacrylate (PMMA), epoxy, or other suitable polymers.

In certain embodiments, as shown, the manifold body 460, e.g., the manifold proximal end 460P of the manifold body 460, can be directly secured and/or coupled to the guide end protector 468. Stated in another manner, in such embodiments, at least a portion of the guide end protector 468 is positioned between the manifold proximal end 460P and the energy guide 422A. Additionally, or in the alternative, at least a portion of the manifold proximal end 460P of the manifold body 460 can be substantially directly secured and/or coupled to the energy guide 422A.

The energy diverter 470 is configured to divert the energy generated within the catheter fluid 232 within the body chamber 466 so that such energy is more accurately directed toward the manifold apertures 462 that are formed into the manifold body 460. The energy diverter 470 can have any suitable size, shape and design for purposes of diverting and directing the energy toward the manifold apertures 462 as desired. In the embodiment illustrated in FIG. 4, the energy diverter 470 is somewhat cone-shaped with a substantially flat, angled outer surface, and is positioned adjacent to the manifold distal end 460D such that the energy is deflected away from the manifold distal end 460D and toward the manifold apertures 462 positioned near the manifold distal end 460D. Additionally, in certain embodiments, the energy diverter 470 can include one or more of a reflecting element, a refracting element, and a fiber diffuser. Alternatively, the energy diverter 470 can have another suitable size, shape or design, or be positioned in a different manner than what is specifically shown in FIG. 4. For example, in some embodiments, the energy diverter 470 can include a convex surface, a concave surface, be somewhat ball-shaped, or have another suitable shape.

FIG. 5 is a schematic cross-sectional view of a portion of the energy guide 522A and yet another embodiment of the energy manifold 529. As shown in FIG. 5, the energy manifold 529 is somewhat similar in design, positioning and function to the previous embodiments. For example, the energy manifold 529 again includes a manifold body 560 including a manifold proximal end 560P that is coupled to and/or secured to the guide distal end 522D of the energy guide 522A; and one or more manifold apertures 562 that are formed into and/or extend through the manifold body 560. In this embodiment, the energy manifold 529 is again configured to direct and concentrate acoustic and/or mechanical energy from the body chamber 566 as defined by the manifold body 560 through the manifold apertures 562 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106.

Similar to the embodiment illustrated in FIG. 4, the energy manifold 529 can again include an energy diverter 570 that is positioned adjacent to the manifold distal end 560D such that energy is deflected away from the sealed, manifold distal end 560D and toward the manifold apertures 562. In this embodiment, the energy diverter 570 is substantially ball-shaped. Alternatively, the energy diverter 570 can have another suitable size, shape or design than that illustrated in FIG. 5.

However, in this embodiment, the energy manifold 529 and/or the energy guide 522A can further include an optical element 572, e.g., a lens or another suitable type of optical element, that is directly coupled to and/or formed directly onto the guide distal end 522D of the energy guide 522A. Additionally, as shown, the optical element 572 can be positioned to extend into the body chamber 566 as defined by the manifold body 560. In some embodiments, the optical element 572 can be an energy-resistant optical element that is configured to focus the energy, e.g., light energy, that is directed from the guide distal end 522D. Additionally, the optical element 572 can further be configured to enhance the energy concentration needed to form the plasma within the catheter fluid 232 (illustrated in FIG. 2) that can be retained within the manifold body 560, i.e. within the body chamber 566. In certain such embodiments, the optical element 572 can be formed from sapphire. Alternatively, the optical element 572 can be formed from one or more other suitable materials.

As shown, in certain implementations, the optical element 572 and a portion of the manifold proximal end 560P can also form a protective enclosure for the guide distal end 522D of the energy guide 522A, i.e. in a manner somewhat similar to the guide end protector 468 illustrated in FIG. 4.

In the embodiment shown in FIG. 5, the body chamber 566 can have a generally tapered design, such that the body chamber 566 is larger and/or wider near the manifold proximal end 560P, the energy guide 522A and the optical element 572, and smaller and/or thinner near the manifold distal end 560D and the manifold apertures 562. With such design, the body chamber 566 can be said to include and/or be segmented into a bubble initiation chamber 556A which is substantially adjacent to the optical element 572 and which is where the plasma bubbles 134 (illustrated in FIG. 1) may be formed within the catheter fluid 232; and a focusing chamber 556B which is substantially adjacent to the manifold distal end 560D and the energy diverter 570 and which is configured to more effectively focus and concentrate the mechanical and/or acoustic energy from the plasma bubbles 134 as they expand toward the manifold distal end 560D. Moreover, the manifold apertures 562, at least some of which are positioned near the manifold distal end 560D is this embodiment, can more effectively concentrate and direct the mechanical and/or acoustic energy of the bubbles 134 outward in a radial pattern toward the specific areas of the balloon wall 230 that are in contact with the vascular lesions 106A at the treatment site 106 to enhance the delivery of such energy to the treatment site 106.

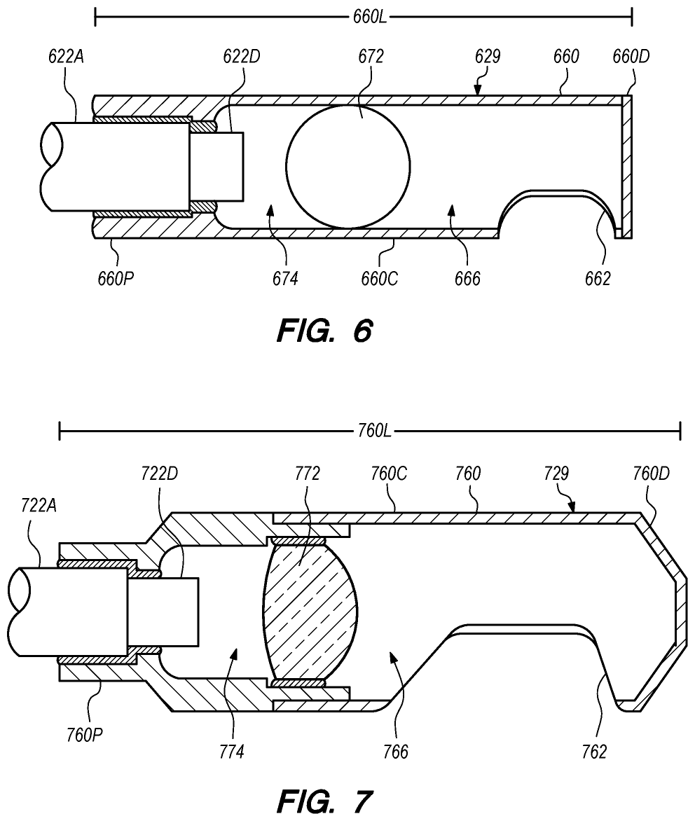

FIG. 6 is a schematic cross-sectional view of a portion of the energy guide 622A and still another embodiment of the energy manifold 629. As shown in FIG. 6, the energy manifold 629 is somewhat similar in design, positioning and function to the previous embodiments. For example, the energy manifold 629 again includes a manifold body 660 including a manifold proximal end 660P that is coupled to and/or secured to the guide distal end 622D of the energy guide 622A; and one or more manifold apertures 662 that are formed into and/or extend through the manifold body 660. In this embodiment, the manifold body 660 includes only a single manifold aperture 662 that is positioned near the substantially flat, sealed, manifold distal end 660D. Alternatively, the energy manifold 629 can include more than one manifold aperture 662 that can be positioned spaced apart along a length 660L of the manifold body 660 and/or radially around a perimeter 660C or circumference of the manifold body 660 in any suitable pattern.

In this embodiment, the manifold body 660 is somewhat thicker in the area where it is coupled and/or secured (bonded) to the guide distal end 622D of the energy guide 622A to provide strain relief. Stated in another manner, as shown, the wall of the manifold body 660 at or near the manifold proximal end 660P and substantially adjacent to the energy guide 622A is somewhat thicker than the remainder of the wall of the manifold body 660.

Additionally, the energy manifold 629 is again configured to direct and concentrate acoustic and/or mechanical energy from the body chamber 666 as defined by the manifold body 660 through the manifold aperture 662 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106.

As shown in FIG. 6, in this embodiment, the energy manifold 629 again includes an optical element 672 that is configured to focus and concentrate the energy that is directed from the guide distal end 622D to form the plasma within the catheter fluid 232 (illustrated in FIG. 2) that can be retained within the manifold body 660, i.e. within the body chamber 666. However, in this embodiment, the optical element 672 is positioned spaced apart a gap from the guide distal end 622D of the energy guide 622A to define an air space 674 between the guide distal end 622D and the optical element 672. In one embodiment, the optical element 672 can be a ball lens that is press fit into the body chamber 666 as defined by the manifold body 660. The press fitting of the optical element 672 within the body chamber 666 can effectively seal the air space 674 from the portion of the body chamber 666 where the catheter fluid 232 is retained. With this design, the sealed air space 674 allows the energy from the energy guide 622A to expand before coupling into the optical element 672 without initiating a plasma in the air space 674. It is appreciated that the region of the body chamber 666 distal to the optical element 672 would be immersed in the catheter fluid 232 for purposes of having the plasma be generated therein. In such embodiment, the optical element 672 can be formed from sapphire. Alternatively, the optical element 672 can have a different design and/or be formed from one or more other suitable materials. Additionally, or in the alternative, in certain non-exclusive embodiments, the air space 674 can be filled with a transparent optical medium such as PMMA, epoxy or the like to couple the energy guide 622A to the optical element 672. Still alternatively, the air space 674 can also include a clear index matching liquid, oil, or another suitable fluid.

FIG. 7 is a schematic cross-sectional view of a portion of the energy guide 722A and yet another embodiment of the energy manifold 729. As shown in FIG. 7, the energy manifold 729 is somewhat similar in design, positioning and function to the previous embodiments. For example, the energy manifold 729 again includes a manifold body 760 including a manifold proximal end 760P that is coupled to and/or secured to the guide distal end 722D of the energy guide 722A; and one or more manifold apertures 762 that are formed into and/or extend through the manifold body 760. In this embodiment, the energy manifold 729 is again configured to direct and concentrate acoustic and/or mechanical energy from the body chamber 766 as defined by the manifold body 760 through the manifold apertures 762 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106.

In this embodiment, the energy manifold 729 includes only a single manifold aperture 762 that is positioned near the angled, sealed, manifold distal end 760D. As shown in this embodiment, the manifold aperture 762 can be somewhat larger and/or wider than in the previous embodiments for purposes of directing the plasma-induced bubbles 134 (illustrated in FIG. 1), i.e. the mechanical and/or acoustic energy of the plasma-induced bubbles 134, in a radial direction outward away from the manifold body 760. More particularly, the shape of the manifold aperture 762 in this embodiment directs the bubbles 134 and mechanical and/or acoustic energy outward in a concentrated, highly directional pattern. Alternatively, the energy manifold 729 can include more than one manifold aperture 762 that can be positioned spaced apart along a length 760L of the manifold body 760 and/or radially around a perimeter 760C or circumference of the manifold body 760 in any suitable pattern. Still alternatively, the manifold distal end 760D can have another suitable design and/or shape than what is shown in FIG. 7.

Additionally, in this embodiment, the manifold body 760 is again somewhat thicker in the area where it is coupled and/or secured (bonded) to the guide distal end 722D of the energy guide 722A. However, the manifold body 760 further has a smaller perimeter 760C or circumference in that area adjacent to the guide distal end 722D, but then tapers outward away from the guide distal end 722D to have a slightly larger perimeter 760C or circumference through the remainder of the manifold body 760. Such design is again utilized to provide strain relief.

As shown in FIG. 7, in this embodiment, the energy manifold 729 again includes an optical element 772 that is configured to focus and concentrate the energy that is directed from the guide distal end 722D to form the plasma within the catheter fluid 232 (illustrated in FIG. 2) that can be retained within the manifold body 760, i.e. within the body chamber 766. Similar to FIG. 6, in this embodiment, the optical element 772 is again positioned spaced apart a gap from the guide distal end 722D of the energy guide 722A to define an air space 774 between the guide distal end 722D and the optical element 772. In one embodiment, the optical element 772 can be a sapphire lens that is bonded to the manifold body 760 to effectively seal the air space 774 from the portion of the body chamber 766 where the catheter fluid 232 is retained. With this design, the sealed air space 774 again allows the energy from the energy guide 722A to expand before coupling into the optical element 772 without initiating a plasma in the air space 774. In such embodiment, the region of the body chamber 766 distal to the optical element 772 would be immersed in the catheter fluid 232 for purposes of having the plasma be generated therein. Alternatively, the optical element 772 can have a different design and/or be formed from one or more other suitable materials. Additionally, or in the alternative, in certain non-exclusive embodiments, the air space 774 can again be filled with a transparent optical medium such as PMMA, epoxy or the like to couple the energy guide 722A to the optical element 772.

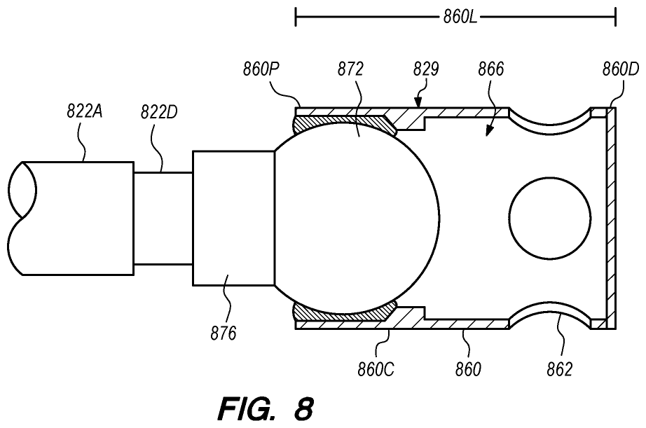

FIG. 8 is a schematic cross-sectional view of a portion of the energy guide 822A and still yet another embodiment of the energy manifold 829. As shown in FIG. 8, the energy manifold 829 is somewhat similar in design, positioning and function to the previous embodiments. For example, the energy manifold 829 again includes a manifold body 860 including a manifold proximal end 860P that is coupled to and/or secured to the guide distal end 822D of the energy guide 822A; and one or more manifold apertures 862 that are formed into and/or extend through the manifold body 860. In this embodiment, the energy manifold 829 includes manifold apertures 862 that are positioned radially about a perimeter 860C or circumference of the manifold body 860 near the substantially flat, sealed, manifold distal end 860D of the manifold body 860. Alternatively, the energy manifold 829 can include any suitable number of manifold apertures 862 that can be positioned spaced apart along a length 860L of the manifold body 860 and/or radially around the perimeter 860C or circumference of the manifold body 860 in any suitable pattern.

In this embodiment, the energy manifold 829 is again configured to direct and concentrate acoustic and/or mechanical energy from the body chamber 866 as defined by the manifold body 860 through the manifold apertures 862 and toward specific areas of the balloon wall 230 (illustrated in FIG. 2) that are in contact with the vascular lesions 106A (illustrated in FIG. 1) at the treatment site 106 (illustrated in FIG. 1) to enhance the delivery of such energy to the treatment site 106.

However, as shown in FIG. 8, the energy manifold 829, i.e. the manifold body 860, is coupled to the energy guide 822A in a different manner than in the previous embodiments. In particular, as illustrated, the energy manifold 829 and/or the energy guide 822A further includes a guide endcap 876 and an optical element 872, e.g., a lens. More specifically, as shown in FIG. 8, the guide endcap 876 is substantially directly coupled to the guide distal end 822D of the energy guide 822A, and the optical element 872 is substantially directly coupled to the guide endcap 876. Additionally, as shown, the manifold body 860, i.e. the manifold proximal end 860P of the manifold body 860, is secured (bonded) to the optical element 872. As such, the body chamber 866 is defined by the manifold body 860 between the optical element 872 and the manifold distal end 860D of the manifold body 860; and the manifold body 860 is positioned spaced apart from the guide distal end 822D of the energy guide 822A.

In certain embodiments, the guide endcap 876 and the optical element 872 can be formed from silica or any other type of glass that can be effectively bonded to the guide distal end 822D of the energy guide 822A. Bonding can be accomplished by fusing glass using a CO₂ laser or an arc discharge source. Alternatively, bonding can be accomplished using polymer adhesives such as UV cured epoxy or acrylates. Still alternatively, bonding can be accomplished in another suitable manner. Yet alternatively, the guide endcap 876 and/or the optical element 872 can be formed from other suitable materials.

As with certain embodiments noted above, the guide endcap 876 and the optical element 872 are configured to focus and concentrate the energy that is directed from the guide distal end 822D to form the plasma within the catheter fluid 232 (illustrated in FIG. 2) that can be retained within the manifold body 860, i.e. within the body chamber 866. Subsequently, the plasma-induced bubbles 134 (illustrated in FIG. 1), i.e. the mechanical and/or acoustic energy of the plasma-induced bubbles 134, can be directed through the manifold apertures 862 outwardly in a radial direction away from the manifold body 860 and toward specific areas of the balloon wall 230 that are in contact with the vascular lesions 106A at the treatment site 106.

FIG. 9A is a schematic cross-sectional view of an alternative embodiment of an energy guide assembly 978A usable within the catheter system 100. In particular, FIG. 9A illustrates that the energy guide assembly 978A includes an energy guide 922A, an extension tube 980A that is coupled to and/or secured to the energy guide 922A, and a plasma generator 933A. Alternatively, the energy guide assembly 978A can include more component or fewer components than those specifically illustrated and described in FIG. 9A.

The energy guide 922A is substantially similar to what has been described in detail previously. As such the energy guide 922A will not be described again in detail. As shown, the energy guide 922A includes a core 986A that is surrounded by a cladding 988A. The core 986A and the cladding 988A of the energy guide 922A can be formed from one or more materials, including but not limited to one or more types of glass, silica, or one or more polymers. The core 986A and the cladding 988A are configured such that the energy (shown as energy beam 990A) from the energy source 124 (illustrated in FIG. 1) is effectively guided along a length of the energy guide 922A from the guide proximal end (not shown in FIG. 9A) to the guide distal end 922D. Additionally, as shown, in some embodiments, the energy guide 922A can further include a guide jacket 964A that is configured to surround and protect the energy guide 922A along a substantial length of the energy guide 922A.

The extension tube 980A is coupled to and/or secured to the energy guide 922A, and extends away from the energy guide 922A. More particularly, as shown, the extension tube 980A can be coupled to and/or secured to the guide distal end 922D of the energy guide 922A, and extends away from the guide distal end 922D of the energy guide 922A. In various embodiments, the extension tube 980A is substantially hollow and is configured to carry some of the catheter fluid 932A that is retained within the balloon interior 146 (illustrated in FIG. 1) of the balloon 104 (illustrated in FIG. 1). In some embodiments, the extension tube 980A includes tube walls 982A that are formed from polymeric non-conductive or dielectric material that surrounds the guide distal end 922D of the energy guide 922A. For example, the extension tube 980A and/or the tube walls 982A can be formed from one or more of Teflon®, polytetrafluoroethylene (PTFE), polyethylene, Kapton®, or other suitable materials.

As shown, it is appreciated that in certain embodiments, the extension tube 980A can further include a tube inlet 992A through which the catheter fluid 932A can enter into the extension tube 980A.

Importantly, in such embodiments, the tube walls 982A of the extension tube 980A have a refractive index at the wavelength of the energy 990A from the energy source 124 that is less than the refractive index of the catheter fluid 932A. For example, in some such embodiments, the catheter fluid 932A can have a refractive index that is between approximately 1.50 and 1.60, and the tube walls 982A of the extension tube 980A can have a refractive index that is between approximately 1.30 and 1.50.

The difference in refractive index between the catheter fluid 932A and the tube walls 982A causes total internal reflection of light incident on an internal surface of the tube wall 982A, directing it back along the axis of the extension tube 980A. The numerical aperture, NA, of such a configuration is given by the formula:

NA=\sqrt{n_{core}²−n_{cladding}²} (Equation 1)

Ideally, the NA for the extension tube 980A would be equal to or greater than that for the energy guide 922A. This would ensure that all of the light energy 990A transmitted to guide distal end 922D of the energy guide 922A would be captured and transmitted to the plasma generator 933A. When the NA of the extension tube 980A is equal to or greater than that for the energy guide 922A, all of the energy 990A entering the extension tube 980A will be captured and transmitted forward, i.e. toward the plasma generator 933A.

The physical behavior of the energy 990A within the extension tube 980A is substantially identical to such behavior within the energy guide 922A itself, except the material inside of the extension tube 980A is fluid and cannot be damaged by the plasma induced bubble or pressure wave. The polymeric or dielectric material is compliant unlike solid materials typically used to form the energy guide 922A. In some instances, such materials of the energy guide 922A can be easily cracked or shattered by acousto-mechanical energy or impingement of high velocity particles. The extension tube 980A can also transmit optical energy very close to the plasma generator 933A thereby increasing its conversion efficiency. The compliance of the material the extension tube 980A is made from and the fact that the main conductor, i.e. the catheter fluid 932A, is liquid enable it to survive the energy from the localized plasma and resulting pressure wave much better than a rigid, frangible material would be able to.

It is appreciated that the energy guide 922A and the extension tube 980A can be of any suitable lengths. For example, in some embodiments, the energy guide 922A can extend substantially to the balloon 104 and the extension tube 980A only extends within the balloon interior 146 of the balloon 104. Alternatively, in other embodiments, the extension tube 980A can be extended in length and potentially become the energy carrier through major portions of the catheter 102 (illustrated in FIG. 1).

The plasma generator 933A is configured to generate plasma when contacted by the energy 990A that has been transmitted through the energy guide 922A and the extension tube 980A. The plasma generator 933A can have any suitable design and/or can be made from any suitable materials. For example, in some embodiments, the plasma generator 933A can be formed from one of metallic or ceramic materials. Alternatively, the plasma generator 933A can be made from other suitable materials.

It is appreciated that by including the extension tube 980A, the guide distal end 922D of the energy guide 922A can be more effectively maintained spaced apart from the plasma that is generated within the catheter fluid 932A. Thus, such design provides a means to improve the durability and longevity of the guide distal end 922D of the energy guide 922A. More specifically, advantages of this approach can include, but are not limited to 1) it moves the guide distal end 922D of the energy guide 922A away from the point where localized plasma is generated thereby minimizing the damaging impact from the bubble and plasma, without degrading performance, 2) it creates a simple means to transmit energy to the plasma generator 933A, 3) it allows the concentrated beam of energy to be transmitted right up to the plasma generator 933A with minimal separation, which increases the conversion efficiency and pressure wave generating capabilities of the energy guide assembly 978A, and 4) it simplifies the design of the plasma generator 933A by reducing dependence on optical and mechanical properties of the energy guide 922A.

In various embodiments, the energy guide assembly 978A is further coupled to an embodiment of the energy manifold such as described in detail herein above. More specifically, the energy guide assembly 978A is usable with any of the embodiments of the energy manifold previously described. Alternatively, in some implementations, the energy guide assembly 978A can be utilized without being coupled to an energy manifold.

FIG. 9B is a schematic cross-sectional view of another alternative embodiment of the energy guide assembly 978B. As illustrated, the energy guide assembly 978B is substantially similar to what was illustrated in the previous embodiment. For example, the energy guide assembly 978B again includes an energy guide 922B and a plasma generator 933B that are substantially similar to the previous embodiment. Additionally, the energy guide assembly 978B again includes an extension tube 980B that is coupled to and/or secured to the guide distal end 922D of the energy guide 922B, and extends away from the guide distal end 922D of the energy guide 922B.

However, in this embodiment, the extension tube 980A is somewhat different than in the previous embodiment. More particularly, in the embodiment shown in FIG. 9B, the tube walls 982B of the extension tube 980B can be formed from a rigid material, such as a metallic or ceramic material, and a dielectric or polymeric coating 984B can be coated onto an inner surface 994B of the tube walls 982B. With such design, the tube walls 982B can provide stronger mechanical structure, and resistance to crushing and damage from plasma and acousto-mechanical energy. The coating 984B on the inner surface 994B of the tube walls 982B can provide the lower refractive index relative to the catheter fluid 932B thereby creating total internal reflectance for the transmitted energy 990B.

It is appreciated that the coating 984B can be added onto the inner surface 994B of the tube walls 982B in any suitable manner. For example, the coating 984B can be added onto the inner surface 994B of the tube walls 982B using solvent film or chemical vapor deposition (CVD). Many options exist to apply uniform thin films to a hard substrate. The basic requirements would be that a thickness of the coating 984B be ten or more times greater than the wavelength of the energy 990B.

In various embodiments, the energy manifold can be utilized to solve many problems that exist in more traditional catheter systems. For example:

1) The energy manifold allows treatment of multiple regions (multiple lesions) within a treatment site that are in contact with a long balloon catheter using a single energy guide, e.g., a single laser pressure wave generator, and eliminates the need to include a plurality of energy guides or a plurality of connected energy sources, e.g., laser energy sources.

2) In traditional catheter systems, the pressure wave energy emitted from the end of a single energy guide or fiber optic source is emitted into a full spherical volume and it therefore contacts a cylindrical region inside a balloon. This may make the single energy guide approach effective for fracturing calcifications only when they are fully circular in cross section. However, the energy manifold concentrates the mechanical energy and localizes it to a specific area by selectively modifying the design of the manifold body, as well as the size, shape and number of manifold apertures in the manifold body of the energy manifold. As a result, this can be much more effective for fracturing lesions that are discontinuous or semi-circular in cross section.

3) In various embodiments, the mechanical assemblage of the energy manifold itself provides a means to protect the guide distal end of the energy guide from the reaction forces and pressure produced by the expanding bubble.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A complete listing of all of the claims in the present Application is as follows:

1. A catheter system for treating a vascular lesion within or adjacent to a blood vessel within a body of a patient, the catheter system comprising:
an energy source that generates energy;
a catheter fluid;
an energy guide including a guide distal end that is selectively positioned near the vascular lesion, the energy guide being configured to receive energy from the energy source and generate a plasma bubble within the catheter fluid; and
an energy manifold that is coupled to the energy guide near the guide distal end, the energy manifold including (i) a manifold body that defines a body chamber, the body chamber being configured to retain at least some of the catheter fluid, and (ii) a manifold aperture that extends through the manifold body;
wherein the energy manifold directs energy from the plasma bubble out of the body chamber through the manifold aperture and toward the vascular lesion.

2. The catheter system of claim 1 wherein the energy manifold includes a plurality of manifold apertures that extend through the manifold body; and wherein the energy manifold is configured to direct energy from the plasma bubble out of the body chamber through each of the plurality of manifold apertures and toward the vascular lesion.

3. The catheter system of claim 2 wherein the plurality of manifold apertures are positioned in a radial pattern around a perimeter of the manifold body.

4. The catheter system of claim 2 wherein the plurality of manifold apertures are arranged in a spiral pattern along a length of the manifold body.

5. The catheter system of claim 2 wherein the plurality of manifold apertures are positioned along a length of the manifold body.

6. The catheter system of claim 1 wherein the energy guide generates one or more pressure waves within the catheter fluid that impart a force upon the vascular lesion.

7. The catheter system of claim 1 wherein the energy guide includes an optical fiber.

8. The catheter system of claim 1 further comprising a balloon including a balloon wall that defines a balloon interior, the balloon being configured to retain the catheter fluid within the balloon interior; and wherein the guide distal end and the energy manifold are positioned within the balloon interior.

9. The catheter system of claim 8 wherein the balloon is selectively inflatable with the catheter fluid to expand to an inflated state, wherein when the balloon is in the inflated state the balloon wall is configured to be positioned substantially adjacent to the vascular lesion.

10. The catheter system of claim 9 wherein the energy manifold is configured to direct energy from the plasma bubble out of the body chamber through the manifold aperture and toward the balloon wall.

11. The catheter system of claim 1 wherein the manifold body includes a manifold proximal end; and wherein the guide distal end of the energy guide is secured to the manifold proximal end of the manifold body.

12. The catheter system of claim 1 wherein the manifold body is substantially cylindrical tube-shaped and defines a substantially cylindrical-shaped body chamber.

13. The catheter system of claim 1 wherein the manifold body includes the manifold proximal end and an opposed manifold distal end; and wherein the body chamber is tapered such that the body chamber is larger near the manifold proximal end and smaller near the manifold distal end.

14. The catheter system of claim 1 further comprising a guide end protector that is coupled to the guide distal end, the guide end protector being configured to protect the guide distal end from energy from the plasma bubble that is generated in the body chamber.

15. The catheter system of claim 1 wherein the energy manifold further includes an energy diverter that diverts energy from the plasma bubble that is generated in the body chamber toward the manifold aperture.

16. The catheter system of claim 15 wherein the manifold body includes a manifold distal end, and wherein the energy diverter is positioned adjacent to the manifold distal end.

17. The catheter system of claim 1 wherein the energy manifold further includes an optical element that is configured to focus the energy that is directed from the guide distal end of the energy guide.

18. The catheter system of claim 17 wherein the optical element is formed from sapphire.

19. The catheter system of claim 17 wherein the optical element is one of directly coupled to and formed directly onto the guide distal end of the energy guide.

20. The catheter system of claim 17 wherein the optical element is positioned spaced apart from the guide distal end of the energy guide to define an air space between the guide distal end and the optical element.

21. The catheter system of claim 20 wherein the air space is sealed from the remainder of the body chamber such that no catheter fluid is retained within the air space.

22. The catheter system of claim 17 further comprising a guide endcap that is directly coupled to the guide distal end of the energy guide; and wherein the optical element is directly coupled to the guide endcap.

23. The catheter system of claim 22 wherein at least one of the guide endcap and the optical element is formed from glass.

24. The catheter system of claim 22 wherein the manifold body includes a manifold proximal end; and wherein the manifold proximal end is secured to the optical element.

25. The catheter system of claim 1 wherein the catheter fluid includes one of a wetting agent and a surfactant.

26. The catheter system of claim 1 further comprising an extension tube that is coupled to and extends away from the guide distal end of the energy guide, the extension tube being configured to retain at least some of the catheter fluid, wherein the energy from the energy source is transmitted through the extension tube after being guided through the energy guide.

27. A method for treating a vascular lesion within or adjacent to a blood vessel within a body of a patient, the method comprising the steps of:
generating energy with an energy source;
positioning a guide distal end of an energy guide near the vascular lesion;
coupling an energy manifold to the energy guide near the guide distal end, the energy manifold including (i) a manifold body that defines a body chamber, the body chamber being configured to retain at least some of a catheter fluid, and (ii) a manifold aperture that extends through the manifold body;
receiving energy from the energy source with the energy guide;
generating a plasma bubble within the catheter fluid with the energy from the energy guide; and
directing energy from the plasma bubble with the energy manifold out of the body chamber through the manifold aperture and toward the vascular lesion.
